(19) United States
(12) Patent Application Publication
(10) Pub. No.: US 2026/0293889 A1
(43) Pub. Date: Oct. 1, 2026

(54) SYNERGISTIC DISINFECTANT FORMULATIONS WITH IMPROVED BIOCIDAL EFFICIENCY AND IMPROVED STABILITY

(71) Applicant: SALVECO, Saint Dié des Vosges (FR)

(72) Inventors: Steve HISIGER, Saint Dié des Vosges (FR); Stephan AUBERGER, Saint Dié des Vosges (FR)

(21) Appl. No.: 19/116,985

(22) PCT Filed: Sep. 28, 2023
(86) PCT No.: PCT/FR2023/051490

§ 371 Date: Mar. 11, 2026

(30) Foreign Application Priority Data
Sep. 29, 2022 (FR) FR2209907

(51) Int. Cl. A01N 37/06; A01N 25/30; A01N 37/02; A01N 37/04; A01P 1/00

(52) U.S. Cl. CPC A01N 37/06; A01N 25/30; A01N 37/02; A01N 37/04; A01P 1/00

(57) ABSTRACT

The invention relates to a biocidal composition comprising: −0.2% to 30% by weight, relative to the total weight of the formulation, of at least one biocidal active substance, the active substance being or comprising a C1-C8 organic acid, in particular an alpha hydroxy carboxylic acid; −0.1% to 30% by weight, relative to the total weight of the formulation, of at least one anionic surfactant, in particular chosen from carboxylates, sulfates or mixtures thereof; −0.09% to 15% by weight, relative to the total weight of the formulation, of non-ionic surfactants comprising:—a first non-ionic surfactant having a (C3-C7) alkyl chain, and—a second non-ionic surfactant having a (C8-C20) alkyl chain; wherein the weight ration expressed as a percentage of the first non-ionic surfactant to the biocidal active substance is between 5% and 200%, in particular between 15% and 100%.

The present invention relates to environmentally friendly biocidal formulations containing an organic acid as active substance, which offer improved biocidal efficiency and good stability over time.

The invention also relates to the use of these formulations for cleaning and disinfecting of hard surfaces or indeed also for ensuring hand hygiene.

### TECHNICAL BACKGROUND

Most biocidal formulations that are intended for cleaning and disinfecting of hands or hard surfaces and have a broad spectrum of activity contain surfactants and antimicrobial active substances.

Surfactants are used, in part, so as to help solubilise any soiling on the skin or hard surfaces, and/or to enhance the wettability of such surfaces or the skin. The surfactants are generally selected from among anionic, non-ionic, amphoteric and/or cationic surfactants.

The antimicrobial active substances commonly used are generally derived as a result of synthetic chemistry and are selected in particular from among quaternary ammoniums, biguanides, triclosan, sodium hypochlorite, etc. However, these active substances pose problems for public health and the environment. In addition to their toxicity to human health and the environment, studies show that they could be the source of growing resistance in microbial species, particularly to antibiotics.

With a view to overcoming these drawbacks, recent years have seen the development of biocidal formulations that are more environmentally friendly and include organic acids by way of active substance.

Thus, the patent EP 2 677 867 B1 provides for biocidal formulations derived from plants for cleaning and disinfecting of surfaces, which comprise by way of active biocidal substance an organic acid, in particular lactic acid and/or citric acid. More particularly, it describes a biodegradable formulation comprising lactic acid, a chelating agent: citric acid, an anionic surfactant of such type as carboxylate or sulfate, and a non-ionic surfactant of such type as C8-C10 or C10-C16 alkyl glycoside, exhibiting biocidal efficacy comparable to that of conventional chemical-synthetic formulations.

However, biocidal formulations derived from plant sources may present compatibility-related problems and therefore offer limited stability over time or inadequate cleaning performance.

Thus, the document US 2010/101,605 discloses multifunctional ‘green’ wipes, which can be activated with water, and which implement three functional compositions:
a cleaning formulation;
a disinfectant formulation; and
a fragrance composition.

However, these functional compositions are implemented in a physically separate manner, in particular by impregnating distinct zones of the wipe, in a manner such as to avoid problems relating to compatibility between the compositions and therefore to the stability of the ingredients over time.

There therefore exists a real need to provide biocidal formulations that provide the means to overcome the drawbacks of biocidal formulations available in the present state of the art, in particular environmentally friendly biocidal formulations that are both efficacious and stable over time.

### SUMMARY OF THE INVENTION

In the current instance, the formulation that has been developed is an environmentally friendly biocidal formulation that has good disinfecting and cleaning properties, combined with good stability over time.

This is achieved by the combining of specific ingredients, in specific content levels, said composition comprising by way of active substance at least one C1-C8 organic acid; at least one anionic surfactant; and at least one combination of non-ionic surfactants: one of which is a short-chain non-ionic surfactant, i.e. the lipophilic part thereof comprises a C3-C7 alkyl chain; and the other of which is a long-chain non-ionic surfactant, i.e. the lipophilic part thereof comprises a C8-C20 alkyl chain.

More particularly, and in a surprising manner, it has been demonstrated that the implemented use of a combination of a short-chain non-ionic surfactant and a long-chain non-ionic surfactant, provided the means, in association with an anionic surfactant, for enhancing the bactericidal and yeasticidal activity of a C1-C8 organic acid, while ensuring good stability of the formulation over time.

Thus, the invention relates firstly to a biocidal formulation comprising:
from 0.2% to 30% by weight, relative to the total weight of the formulation, of at least one biocidal active substance, the active substance being or comprising a C1-C8 organic acid, in particular an alpha hydroxy carboxylic acid;
from 0.1% to 30% by weight, relative to the total weight of the formulation, of at least one anionic surfactant, in particular chosen from carboxylates, sulfates or mixtures thereof;
from 0.09% to 15% by weight, relative to the total weight of the formulation, of non-ionic surfactants comprising:
a first non-ionic surfactant having a (C3-C7)alkyl chain; and
a second non-ionic surfactant having a (C8-C20)alkyl chain;
wherein:
the weight ratio expressed as a percentage of the said first non-ionic surfactant to the said biocidal active substance is between 5% and 200%, in particular between 15 and 100%.

Other advantageous characteristic features of the method according to the invention are specified below:
the C1-C8 organic acid is selected from among formic acid, lactic acid, citric acid, tartaric acid, glycolic acid, malic acid, maleic acid, fumaric acid, adipic acid, and succinic acid, or a mixture thereof; in particular citric acid, lactic acid, tartaric acid, and malic acid, or a mixture thereof;
the non-ionic surfactants are selected from among alkylpolyglycosides, ethoxylated or non-ethoxylated sorbitan esters, ethoxylated or non-ethoxylated polyglycerol esters, ethoxylated alcohols, ethoxylated oils, or mixtures thereof;
the first and/or the second non-ionic surfactant is/are an alkylpolyglycoside;
the said first non-ionic surfactant is a (C3-C7)alkylpolyglycoside, in particular a (C5-C6)alkylpolyglycoside;
the first non-ionic surfactant is a D-xylopyranose (C3-C7)alkyl glycoside, in particular D-xylopyranose 3-methylbutyl glycoside;
the second non-ionic surfactant is a (C8-C20) alkylpolyglycoside, in particular a (C8-C16) alkylpolyglycoside;
the weight ratio expressed as a percentage of the second non-ionic surfactant to the said biocidal active substance is between 0.5 and 200%, in particular between 2 and 50%;
the first non-ionic surfactant represents from 15% to 90% by weight, in particular from 25% to 55% by weight of the total weight of the non-ionic surfactants present in the formulation;
the weight ratio expressed as a percentage of the said first surfactant to the said second non-ionic surfactant is between 15 and 500%, in particular from 55% to 200%;
the weight ratio expressed as a percentage of the non-ionic surfactants to the said active substance is between 5 and 200%, and more particularly between 15% and 100%;
the weight ratio expressed as a percentage of the said anionic surfactant to the said biocidal active substance is between 20% and 200%, in particular between 50% and 100%;
the anionic surfactant comprises at least one surfactant of carboxylate type, in particular 2-(2-octoxyethoxy) acetic acid or the sodium salt thereof;
the anionic surfactant comprises an anionic surfactant of sulfate type, in particular having a C8-C20 alkyl or alkoxy chain, in particular sodium lauryl sulfate; or a sodium (C8-C16)alkyl ether sulfate;
the biocidal active substance of the formulation is constituted of a single organic acid, which is either lactic acid or citric acid, preferably lactic acid;
the formulation further comprises a pH adjusting agent, a thickening agent, a colouring agent, an emollient, a fragrance, or a mixture thereof;
the formulation is a so-called concentrated formulation and comprises:
from 5% to 30% by weight relative to the total weight of the formulation of at least one biocidal active substance, the active substance being or comprising a C1-C8 organic acid, in particular an alpha hydroxy carboxylic acid;
from 5% to 30% by weight relative to the total weight of the formulation, in particular from 15% to 30% by weight, of at least one anionic surfactant, in particular chosen from carboxylates, sulfates or mixtures thereof;
from 1% to 12% by weight relative to the total weight of the formulation, in particular from 2% to 8% by weight of non-ionic surfactants, comprising:
a first non-ionic surfactant having a (C3-C7)alkyl chain;
a second non-ionic surfactant having a (C8-C20)alkyl chain;
the formulation is ready to use and comprises:
from 0.2% to 2.5% by weight relative to the total weight of the formulation of at least one biocidal active substance, the active substance being a C1-C8 organic acid, in particular an alpha hydroxy carboxylic acid;
from 0.1% to 5% by weight relative to the total weight of the formulation, in particular from 0.3% to 2% by weight, of at least one anionic surfactant, in particular chosen from carboxylates, sulfates or mixtures thereof;
from 0.08% to 4%, preferably from 0.09% to 4% by weight relative to the total weight of the formulation, in particular from 0.1% to 2% by weight of non-ionic surfactants, comprising:
a first non-ionic surfactant having a (C3-C7)alkyl chain;
a second non-ionic surfactant having a (C8-C20)alkyl chain;
the first non-ionic surfactant represents from 0.009% to 1.8% by weight, in particular from 0.02% to 0.9% by weight relative to the total weight of the formulation;
according to this last embodiment, the second non-ionic surfactant represents from 0.08% to 2% by weight relative to the total weight of the formulation; a. the ready to use formulation is obtained by the diluting of a concentrated formulation with water, in particular in a ratio: of 0.1% to 25% by weight (of the concentrated formulation in water), in particular of 0.5% to 10% by weight, more specifically of 0.5% to 5% by weight;
more than 50% by weight, preferably more than 75% by weight, in particular more than 95% by weight of the ingredients of the formulation are derived from renewable sources;
the formulation is more than 95% biodegradable, in particular more than 98% biodegradable in accordance with the OECD 301 Guidelines.

In accordance with another aspect, the invention also relates to the use of a biocidal formulation according to the invention, for cleaning and disinfecting inanimate hard surfaces, including surfaces that come into contact with food; for human hygiene, in particular for the sanitising and washing of hands; for cleaning and disinfecting animal teats before or after milking; and for washing and disinfecting fruits and vegetables.

### BRIEF DESCRIPTION OF THE FIGURES

FIG. 1 represents a graph that reports results of the evaluation of the degreasing cleaning power of the formulations tested. The y-axis corresponds to the ratings assigned to the formulations.

FIG. 2 represents photos of the bottles of the tested formulations that enable assessing the stability of the formulations.

### DETAILED DESCRIPTION

The invention is hereinafter disclosed in greater detail and in a non-limiting manner in the description that follows.

Unless otherwise indicated, all percentages relating to quantities are percentages by weight.

The percentages by weight indicated for the ingredients of the biocidal formulation are expressed relative to the total weight of the formulation.

The weight ratios are expressed on a percentage basis.

For the purposes of the present application the term ‘biocidal formulation’ is understood to refer to a formulation that has at least bactericidal activity, and preferably both bactericidal and yeasticidal activity.

The term ‘bactericidal activity’ is understood to refer to a formulation that is capable of satisfying the requirements of the standard EN 1276:2019 at 20° C. against bacteria, in particular against one or more of the following: Escherichia coli, Staphylococcus aureus, Enterococcus hirae, and Pseudomonas aeruginosa; more particularly against at least two, in particular at least three; more particularly against all four of these microorganisms; i.e. a microbial logarithmic reduction of at least 5 on contact with the formulation, within a maximum period of 5 minutes. In preferred embodiments, this requirement is satisfied within 2 minutes, and in certain embodiments, more specifically within one minute. In certain specific embodiments, it has been found that for at least certain organisms, satisfying the requirements according to the standard EN 1276 is achieved within merely 30 seconds.

The term “yeasticidal activity” is understood to refer to a formulation that is capable of satisfying the requirements according to the standard EN 1650:2019 at 20° C., i.e. of achieving a microbial log reduction of at least 4 under soiling conditions within a maximum period of 5 minutes. In preferred embodiments, this requirement is satisfied within 1 minute, and in certain embodiments, more specifically within 30 seconds.

The term ‘cleaning power’ is understood to refer to formulations capable of cleaning and solubilising greasy soiling or residues deposited on a hard surface. It was evaluated in accordance with the method proposed by the ‘German Cosmetic, Toiletry, Perfumery and Detergent Association (IKW)’ and published in SOFW Journal, Volume 144 (2018), with the modifications indicated.

The term ‘ingredients derived from renewable sources’ or ‘ingredients derived from natural sources’ is understood to refer to ingredients that comprise carbon from renewable sources.

In fact, unlike ingredients derived from fossil materials, ingredients derived from renewable raw materials contain ¹⁴C. All carbon samples taken from living organisms (animals or plants) are in fact a mixture of 3 isotopes: ¹²C (representing approximately 98.892%), ¹³C (approximately 1.108%) and ¹⁴C (traces: 1.2×10-12%). The ¹⁴C/¹²C ratio in living tissue is identical to that in the atmosphere. In the environment, ¹⁴C exists in two predominant forms: in mineral form, i.e. carbon dioxide (CO₂), and in organic form, i.e. carbon integrated into organic molecules.

In a living organism, the ¹⁴C/¹²C ratio is maintained constant by metabolism because carbon is continually exchanged with the environment. Given that the proportion of ¹⁴C is constant in the atmosphere, the same applies to the organism, as long as it is living, since it absorbs this ¹⁴C in the same way as it absorbs ¹²C. The average ¹⁴C/¹²C ratio is equal to 1.2×10-12.

¹²C is stable, that is to say the number of ¹²C atoms in a given sample is constant over time. ¹⁴C, on the other hand, is radioactive (each gram of living carbon contains enough ¹⁴C isotope to give 13.6 disintegrations per minute) and the number of such atoms in a sample decreases over time (t) according to the law:
n=no exp (−at); where:
   no is the initial number of ¹⁴C atoms present (at the death of the creature, animal or plant);
n is the number of ¹⁴C atoms remaining at the end of time t;
a is the decay constant (or radioactive constant), it is related to the half-life of ¹⁴C.

The half-life (or period) is the time period required for any number/amount of radioactive nuclei or unstable particles of a given species to be reduced to half the initial value through decay; the half-life T½ is related to the decay constant a by the formula a T½=In 2. The half-life of ¹⁴C is equal to 5730 years.

Given the half-life (T½) of ¹⁴C, it is considered that the ¹⁴C content is constant from the extraction of the plant raw materials to the manufacture of the formulation, and even until the conclusion of use thereof.

The applicant considers an ingredient to be derived from renewable raw materials if it contains at least 20% by mass of C from renewable sources relative to the total mass of carbon, preferably at least 50% by mass of C from renewable sources relative to the total mass of carbon.

In other words, an ingredient is derived from renewable raw materials if it contains at least 0.2×10-10% by mass of ¹⁴C, preferably 0.6×10-10% by mass of ¹⁴C.

At present, there are at least two different techniques for measuring the ¹⁴C content of a sample as follows:
Liquid scintillation spectrometry: This method consists of counting the ‘Beta’ particles resulting from the decay of ¹⁴C: the Beta radiation emitted by a sample of known mass (known number of ¹²C atoms) is measured over a certain period of time. This ‘radioactivity’ is proportional to the number of ¹⁴C atoms, which may be determined in this manner. The ¹⁴C present in the sample emits β-radiation, which upon contact with the scintillating liquid (scintillator) leads to the generation of photons. These photons have different energies (between 0 and 156 Kev) and form what is known as a ¹⁴C spectrum. According to two variants of this method, the analysis is carried out either on CO₂ previously produced by the carbonaceous sample in an appropriate absorbent solution, or on benzene after prior conversion of the carbonaceous sample into benzene.
Mass spectrometry: The sample is reduced to graphite or gaseous CO₂ and analysed in a mass spectrometer. This technique uses an accelerator and a mass spectrometer in order to separate the ¹⁴C ions from the ¹²C ions and thus to determine the ratio between the two isotopes.

All of these methods for measuring the ¹⁴C content of materials are described in detail in the standards ASTM D 6866 (in particular D6866-06) and ASTMD 7026 (in particular 7026-04). These methods measure the ¹⁴C/¹²C ratio of a sample and compare it with the ¹⁴C/¹²C ratio of a reference sample derived from 100% renewable sources, in order to provide a relative percentage of C derived from renewable sources in the sample.

The measurement method preferentially used in the case of the ingredients of the formulation of the invention is mass spectrometry as described in the standard ASTM D6866-06 (“accelerator mass spectroscopy”).

For the purposes of this description, the term ‘biodegradable’ is understood to refer to an organic substance capable of undergoing biodegradation based on a biological process of degradation into smaller, simpler molecules. Thus, degradation in the presence of oxygen leads to the production of CO₂, water and minerals, while anaerobic biodegradation produces for the most part methane. The rate of biodegradability of the biocidal formulation represents the percentage by weight of biodegradable ingredients relative to the total weight of the formulation in dry mass, i.e. without water, and may be determined in accordance with the OECD 301 Guidelines.

The term ‘stable biocidal formulations’ is understood to refer to biocidal formulations capable of retaining their biocidal and cleaning efficacy for a period of more than 2 years at ambient temperature. This stability may be assessed in accordance with the CIPAC MT46.3 method under accelerated ageing conditions, in particular by increasing the temperature of the samples.

### Biocidal Formulation

Thus, according to a first aspect, the invention relates to a biocidal formulation comprising:
from 0.2% to 30% by weight of at least one biocidal active substance, the active substance being or comprising a C1-C8 organic acid, in particular an alpha hydroxy carboxylic acid;
from 0.1% to 30% by weight, of at least one anionic surfactant, in particular chosen from carboxylates, sulfates or mixtures thereof;
from 0.09% to 15% by weight of non-ionic surfactants comprising:
a first non-ionic surfactant having a (C3-C7)alkyl chain; and
a second non-ionic surfactant having a (C8-C20)alkyl chain;
wherein the weight ratio of the said first non-ionic surfactant to the said biocidal active substance is between 5% and 200%, in particular between 15 and 100%.

The biocidal formulations according to the invention advantageously exhibit bactericidal activity. Preferably, they are both bactericidal and yeasticidal. They additionally also possess cleaning and degreasing power.

In some embodiments, more than 50% by weight, preferably more than 75% by weight, in particular more than 95% by weight of the ingredients of the formulation are derived from renewable sources.

In some embodiments, the formulation is more than 95% biodegradable, in particular more than 98% biodegradable in accordance with OECD guidelines, in particular OECD 301.

### Active Substance

The formulations according to the invention comprise at least one active substance, the said active substance being or comprising a C1-C8 organic acid.

For the purposes of this description, the range of values from “x to y % by weight” is applicable to the total content of active substance in the formulation, and therefore does not apply solely to the organic acids present in the formulation.

In some embodiments, the biocidal formulations according to the invention do not comprise biocidal active substances other than C1-C8 organic acids. In other words, the biocidal active substance of the formulation is selected from among C1-C8 organic acids and mixtures thereof, and thus comprises no other (particularly, chemical-synthetic) biocidal active substance.

In some embodiments, the biocidal formulations according to the invention do not comprise alcohol, in particular ethanol.

The C1-C8 organic acid may be selected in particular from among formic acid, lactic acid, citric acid, tartaric acid, glycolic acid, malic acid, maleic acid, fumaric acid, adipic acid, and succinic acid, or a mixture thereof.

These organic acids may advantageously be biodegradable and/or be derived from renewable (in particular plant-based) resources.

In a preferred manner, the organic acid is an alpha hydroxy carboxylic acid, i.e. a carboxylic acid comprising at least one hydroxyl functional group in the alpha position. Preferably, the organic acid is selected from among citric acid, lactic acid, tartaric acid, and malic acid, or a mixture thereof.

In some embodiments, the biocidal active substance of the formulation is constituted of a single organic acid which is either lactic acid or citric acid, preferably lactic acid.

### Non-Ionic Surfactants

In some embodiments, the non-ionic surfactants are selected from among alkylpolyglycosides, ethoxylated or non-ethoxylated sorbitan esters, ethoxylated or non-ethoxylated polyglycerol esters, ethoxylated alcohols, ethoxylated plant oils, or mixtures thereof.

Depending on the length of the alkyl chain, the surfactants may be classified under the first or second category of non-ionic surfactants. This pertains to the length of the alkyl chain of: the alkyl group in alkyl polyglycoside; the alkyl group of the acid part in sorbitan esters, whether or not ethoxylated; the alkyl group of the acid part in polyglycerol esters, whether or not ethoxylated; the alkyl group of ethoxylated alcohol. With regard to ethoxylated oils, these belong to the family of non-ionic surfactants having a (C8-C20)alkyl chain.

Advantageously, these non-ionic surfactants may be biodegradable and/or derived from renewable (in particular plant-based) resources.

In some embodiments, the first and/or the second non-ionic surfactant is/are an alkylpolyglycoside also known as APG. An alkylpolyglycoside comprises a polymer chain that includes a sugar unit containing an alkoxy functional group. The sugar unit may be selected from among glucose, dextrose, sucrose, fructose, galactose, maltose, maltotriose, lactose, cellobiose, mannose, ribose, dextran, talose, allose, xylose, levoglucan and D-xylopyranose. The alkyl chain of the alkoxy group may be linear or branched. With respect to the first surfactant, it comprises 3 to 7 carbon atoms, that is to say propyl, butyl, pentyl, hexyl or heptyl chains; and with respect to the second surfactant, it comprises between 8 and 20 carbon atoms, that is to say, octyl (caprylyl), nonyl, decyl (capryl), undecyl, dodecyl (lauryl), myristyl, palmityl, stearyl and arachidic chains.

In some embodiments, the said first non-ionic surfactant is a (C3-C7) alkylpolyglycoside, in particular a (C5-C6)alkylpolyglycoside.

In some embodiments, the first non-ionic surfactant is a D-xylopyranose (C3-C7)alkyl glycoside, in particular D-xylopyranose 3-methylbutyl glycoside.

In some embodiments, the second non-ionic surfactant is a (C8-C20) alkylpolyglycoside, in particular a (C8/C18) alkylpolyglycoside, and in particular a (C8-C16) alkylpolyglycoside.

A mixture of APGs of various alkyl chain lengths is generally used. They are denoted as Ca/Cb, with a and b denoting the number of carbon atoms in the alkyl chain. The following mixtures are particularly suitable: a (C8/C10)alkyl polyglycoside, a (C10/C12)alkyl polyglycoside, or a (C10/C16)alkyl polyglycoside.

In some embodiments, the weight ratio of the second non-ionic surfactant to the said biocidal active substance is between 0.5 and 200%, in particular between 2 and 50%.

In some embodiments, the first non-ionic surfactant represents from 15% to 90% by weight, in particular from 25% to 55% by weight of the total weight of the non-ionic surfactants present in the formulation.

In some embodiments, the weight ratio of the said first surfactant to the said second non-ionic surfactant is between 15 and 500%, in particular from 55% to 200%.

In some embodiments, the weight ratio of non-ionic surfactants to the said active substance is between 5 and 200%, and more particularly between 15% and 100%.

### Anionic Surfactants

The biocidal formulations according to the invention comprise an anionic surfactant.

In some embodiments, the weight ratio of the said anionic surfactant to the said biocidal active substance is between 20% and 200%, in particular between 50% and 100%.

Preferably, the anionic surfactant is selected from among anionic surfactants that are biodegradable and/or derived from renewable (in particular plant-based) resources.

In some embodiments, the anionic surfactant comprises at least one surfactant of carboxylate type, in particular 2-(2-octoxyethoxy) acetic acid or the sodium salt thereof.

In some embodiments, the anionic surfactant comprises an anionic surfactant of sulfate type, in particular having a C8-C20 alkyl or alkoxy chain, in particular sodium lauryl sulfate; or a sodium (C8-C16)alkyl ether sulfate.

### Other Ingredients

The biocidal formulation according to the invention may further comprise a pH adjusting agent, a thickening agent, a colouring agent, an emollient, a fragrance, or a mixture thereof.

These ingredients may represent a total of 0% to 3% of the total weight of the formulation.

In some embodiments, the biocidal formulation according to the invention comprises from 25% to 99.1%, in particular from 35 to 98% of water.

### Diluted, Ready to Use Biocidal Formulations

The formulations according to the invention may include concentrated formulations, to be diluted in water so as to form ready to use formulations, or solutions for use that provide the means for obtaining the desired cleaning and biocidal efficacy when brought into direct contact with the surface to be cleaned.

In some embodiments, the formulation comprises:
from 0.2% to 2.5% by weight of at least one biocidal active substance, the active substance being a C1-C8 organic acid, in particular an alpha hydroxy carboxylic acid;—from 0.1% to 5% by weight, in particular from 0.3% to 2% by weight, of at least one anionic surfactant, in particular chosen from carboxylates, sulfates or mixtures thereof;
from 0.08% to 4% by weight, preferably from 0.09% to 4%, in particular from 0.1% to 2% by weight of non-ionic surfactants, comprising:
a first non-ionic surfactant having a (C3-C7)alkyl chain,
a second non-ionic surfactant having a (C8-C20)alkyl chain;
the said formulation being a ready to use formulation.

In some embodiments of the ready to use formulation, the first non-ionic surfactant represents from 0.009% to 1.8% by weight, in particular from 0.02% to 0.9% by weight of the total weight of the formulation.

In some embodiments, the second non-ionic surfactant represents from 0.08% to 2% by weight of the total weight of the formulation.

In some embodiments, the ready to use formulation is obtained by the diluting of a more concentrated formulation with water, in particular in a ratio: of 0.1% to 25% by weight (of the concentrated formulation in water), in particular of 0.5% to 10% by weight, more specifically of 0.5% to 5% by weight.

### Concentrated Biocidal Forumations, to be Diluted

In some embodiments, the formulation comprises:
from 5% to 30% by weight of at least one biocidal active substance, the active substance being or comprising a C1-C8 organic acid, in particular an alpha hydroxy carboxylic acid;
from 5% to 30% by weight, in particular from 15% to 30% by weight, of at least one anionic surfactant, in particular chosen from carboxylates, sulfates or mixtures thereof;
from 1% to 12% by weight, in particular from 2% to 8% by weight of non-ionic surfactants, comprising:
a first non-ionic surfactant having a (C3-C7)alkyl chain;
a second non-ionic surfactant having a (C8-C20)alkyl chain;
the said formulation being a concentrated formulation.

### Use of Biocidal Formulations

According to a second aspect, the invention relates to the use of a biocidal formulation according to the invention, for cleaning and disinfecting inanimate hard surfaces, including surfaces that come into contact with food; for human hygiene, in particular for the sanitising and washing of hands; for cleaning and disinfecting animal teats before or after milking; and for washing and disinfecting fruits and vegetables.

### Examples

### Preparation of Formulations

The formulations are prepared by the adding of the different ingredients to fresh water (6° TH) at 22° C. (+/−3° C.) in accordance with the following Table 1.

TABLE 1

| | Formulation | Formulation | Formulation | Formulation |
|---|---|---|---|---|
| | A | B | C | D |
| | comparative | comparative | comparative | comparative |
| Water (% w/w) | 98.9 | 96.9 | 96.9 | 96.9 |
| Lactic Acid | 0.6 | 0.6 | 0.6 | 0.6 |
| (% w/w) | | | | |
| Anionic | 0.4 | 0.4 | 0.4 | 0.4 |
| Surfactant | | | | |
| (% w/w) | | | | |
| C12 APG | 0 | 2 | 0 | 0.75 |
| (% w/w) | | | | |
| C5 APG | 0 | 0 | 2 | 1.25 |
| (% w/w) | | | | |
| Fragrance | 0.1 | 0.1 | 0.1 | 0.1 |
| (% w/w) | | | | |

Evaluation is performed of the biocidal activity, the degreasing cleaning power, and the stability of the formulations.

### Biocidal Activity

The formulations are tested in accordance with the French standard NF EN 1276 (2019) in order to measure bactericidal activity, and in accordance with the French standard NF EN 1650 (2019) in order to measure yeasticidal activity.

The formulations are ready to use and evaluated at a concentration of 80% (v/v) as recommended in the protocols of the corresponding standards. The tests are carried out with model strains of Escherichia coli ATCC 10536, Staphylococcus aureus ATCC 6538, Pseudomonas aeruginosa ATCC 15442, Enterococcus hirae ATCC 10541 for bactericidal activity; as well as the strain Candida albicans ATCC 10231 for yeasticidal activity.

The strains are brought into contact with an interfering substance, bovine albumin (fraction V) at 3 g/L, which represents the fouling conditions defined in the 2 standards.

The biocidal formulation is then added thereto and left in contact with the microbial strain/interfering substance mixture for a period of 5 minutes (+10 seconds) at 20° C. (+1° C.). At the end of the contact time period, the biocidal activity is neutralised by using the dilution/neutralisation method. The neutraliser is prepared in distilled water and is composed of sodium thiosulphate at 5 g/L, soya lecithin at 3 g/L, polysorbate 80 at 30 g/L, and phosphate buffer (0.25 mol/L) at 100 g/L. Using the spread plate technique consisting of thorough and even spreading of the neutralised mixture over the medium, the surviving microorganisms are counted after an incubation period of 24 h at 37° C. (+1° C.) for bactericidal activity, and 48 h at 30° C. (+1° C.) for yeasticidal activity (using TSA and GEM media respectively), in order to calculate a logarithmic reduction relative to the initial concentration of microorganisms in the test. The results are reported in Table 2 below.

TABLE 2

| | Formulation | Formulation | Formulation | Formulation |
|---|---|---|---|---|
| | A | B | C | D |
| Bactericidal | ++ | ++ | ++ | ++ |
| Activity | | | | |
| Yeasticidal | ++ | + | ++ | ++ |
| Activity | | | | |

In Table 2:
the value ++ indicates a microbial logarithmic reduction greater than 5,
the value + indicates a microbial logarithmic reduction greater than 3.

The results show that the formulations tested satisfy the acceptance criteria of the standard EN 1276:2019. The formulations A, C and D thus satisfy the acceptance criteria of the standard EN 1650:2019.

These results show that formulation B comprising a long-chain non-ionic surfactant, as the only non-ionic surfactant in the formulation, exhibits less efficacious yeasticidal activity than the activities observed for formulations A, C and D. As a consequence, the formulation B was excluded from further testing.

The degreasing cleaning power and stability of formulations A, C and D, exhibiting enhanced biocidal activity, were subsequently evaluated.

### Degreasing Cleaning Power:

The degreasing cleaning power of the different formulations was assessed in accordance with the so-called ‘abrasimeter’ method as described by the ‘German Cosmetic, Toiletry, Perfumery and Detergent Association (IKW)’, and published in SOFW Journal, Volume 144 (2018), with the following modifications:

The support substrates used consist of glass plates measuring 46×33 cm and covered with a glossy white adhesive film.

The surface is first pre-cleaned with a 70% (v/v) ethanol solution and then left to dry for 1 hour.

Applied over the surface thereafter is a mixture of soiling substances. This consists of a coloured hydroalcoholic composition derived from fatty and proteinaceous based foods.

The mixture of soiling substances is heated with slow agitation to 70° C. for a period of 10 minutes before being cooled to ambient temperature.

The soiling substance is heated to 50° C. before being uniformly applied (3 mg/cm2) using a foam roller, over the entire surface to be tested.

The same is allowed to dry for a period of 12 hours at ambient temperature (22° C.+/−3° C.).

The surfaces are then cleaned with an abrasimeter (TQC Dry Washability Tester-model AB5210) using sponges (Spontex® Azela 86 9×4 cm) that have been previously rinsed 3 times with clean water, and thereafter uniformly impregnated with 30 g of the product to be tested.

A control check is carried out by replacing the product to be tested with water (6° TH).

The sponges are positioned on the abrasimeter and a weight of 100 g is applied to each sponge in accordance with the manufacturer's instructions.

The cleaning is carried out using the abrasimeter over 5 return cycles, at a speed of 35 cycles/min.

The assessment of the cleaning power is carried out by means of a process of visual rating from 0 to 5 according to a pre-defined scale, with a rating of 5 indicating the total elimination of soiling, and a rating of 0 indicating an elimination level similar to that of the water control. The tests are repeated at least 5 times for each product tested in order to be able to calculate the average results for each product.

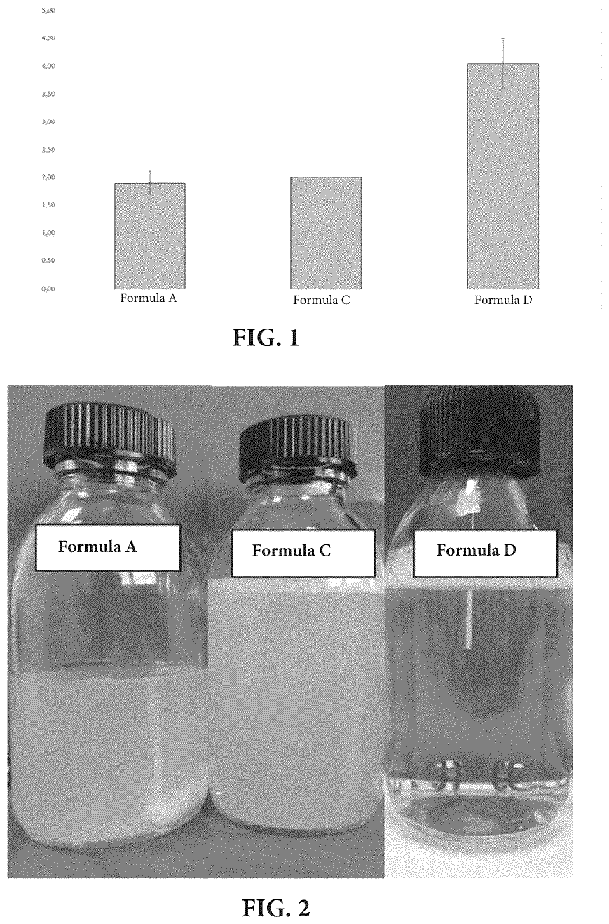

The results are reported in FIG. 1. The results show that the formulation D according to the invention exhibits a cleaning and degreasing power at least 50% greater than the comparative formulations A and C.

### Stability

The stability of the formulations is assessed in accordance with the method CIPAC MT 46.3 ‘Accelerated Storage Procedure’ by placing the different formulations in glass bottles for a period of 14 days at 54° C. (+/−1° C.). The appearance of the formulations is then assessed visually upon the samples returning to ambient temperature (22+/−3° C.).

The results are reported in FIG. 2. The bottles have been photographed. The results show that the formulation according to invention D is clear, unlike the formulations A and C, which are opaque.

In conclusion, the formulation according to the invention comprising the said combination of non-ionic surfactants exhibits enhanced biocidal activity as compared to formulation B, which by way of non-ionic surfactant, comprises only a long-chain non-ionic surfactant. In addition, the formulation according to the invention exhibits significantly enhanced degreasing cleaning power and stability as compared to formulation A, which does not comprise a non-ionic surfactant; and as compared to formulation C, which comprises an anionic surfactant and a short-chain non-ionic surfactant.

Thus, the presence of a combination of specific non-ionic surfactants provides the means for enhancing each of the properties tested.

1. A biocidal formulation comprising:
from 0.2% to 30% by weight, relative to the total weight of the formulation, of at least one biocidal active substance, the active substance being or comprising a C1-C8 organic acid, in particular an alpha hydroxy carboxylic acid;
from 0.1% to 30% by weight, relative to the total weight of the formulation, of at least one anionic surfactant, in particular chosen from carboxylates, sulfates or mixtures thereof;
from 0.09% to 15% by weight, relative to the total weight of the formulation, of non-ionic surfactants comprising:
a first non-ionic surfactant having a (C3-C7)alkyl chain; and
a second non-ionic surfactant having a (C8-C20)alkyl chain;
wherein:
the weight ratio expressed as a percentage of the said first non-ionic surfactant to the said biocidal active substance is between 5% and 200%, in particular between 15 and 100%.

2. A formulation according to claim 1, wherein the C1-C8 organic acid is selected from among formic acid, lactic acid, citric acid, tartaric acid, glycolic acid, malic acid, maleic acid, fumaric acid, adipic acid, and succinic acid, or a mixture thereof; in particular citric acid, lactic acid, tartaric acid, and malic acid, or a mixture thereof.

3. A formulation according to claim 1 or 2, wherein the non-ionic surfactants are selected from among alkylpolyglycosides, ethoxylated or non-ethoxylated sorbitan esters, ethoxylated or non-ethoxylated polyglycerol esters, ethoxylated alcohols, ethoxylated oils, or mixtures thereof.

4. A formulation according to claims 1 to 3, wherein the first and/or the second non-ionic surfactant is/are an alkylpolyglycoside.

5. A formulation according to any one of the preceding claims, wherein the said first non-ionic surfactant is a (C3-C7)alkylpolyglycoside, in particular a (C5-C6) alkylpolyglycoside.

6. A formulation according to claim 5, wherein the first non-ionic surfactant is a D-xylopyranose (C3-C7)alkyl glycoside, in particular D-xylopyranose 3-methylbutyl glycoside.

7. A formulation according to any one of the preceding claims, wherein the second non-ionic surfactant is a (C8-C20) alkylpolyglycoside, in particular a (C8-C16) alkylpolyglycoside.

8. A formulation according to any one of the preceding claims, wherein the weight ratio expressed as a percentage of the second non-ionic surfactant to the said biocidal active substance is between 0.5 and 200%, in particular between 2 and 50%.

9. A formulation according to any one of the preceding claims, wherein the first non-ionic surfactant represents from 15% to 90% by weight, in particular from 25% to 55% by weight of the total weight of the non-ionic surfactants present in the formulation.

10. A formulation according to any one of the preceding claims, wherein the weight ratio expressed as a percentage of the said first surfactant to the said second non-ionic surfactant is between 15 and 500%, in particular from 55% to 200%.

11. A formulation according to any one of the preceding claims, wherein the weight ratio expressed as a percentage of the non-ionic surfactants to the said active substance is between 5 and 200%, and more particularly between 15% and 100%.

12. A formulation according to any one of the preceding claims, wherein the weight ratio expressed as a percentage of the said anionic surfactant to the said biocidal active substance is between 20% and 200%, in particular between 50% and 100%.

13. A formulation according to any one of the preceding claims, wherein the anionic surfactant comprises at least one surfactant of carboxylate type, in particular 2-(2-octoxyethoxy) acetic acid or the sodium salt thereof.

14. A formulation according to any one of the preceding claims, wherein the anionic surfactant comprises an anionic surfactant of sulfate type, in particular having a C8-C20 alkyl or alkoxy chain, in particular sodium lauryl sulfate; or a sodium (C8-C16) alkyl ether sulfate.

15. A formulation according to any one of the preceding claims, wherein the biocidal active substance of the formulation is constituted of a single organic acid, which is either lactic acid or citric acid, preferably lactic acid.

16. A formulation according to any preceding claim, wherein the formulation further comprises a pH adjusting agent, a thickening agent, a colouring agent, an emollient, a fragrance, or a mixture thereof.

17. A formulation according to any one of claims 1 to 16, comprising:
from 5% to 30% by weight relative to the total weight of the formulation of at least one biocidal active substance, the active substance being or comprising a C1-C8 organic acid, in particular an alpha hydroxy carboxylic acid;
from 5% to 30% by weight relative to the total weight of the formulation, in particular from 15% to 30% by weight, of at least one anionic surfactant, in particular chosen from carboxylates, sulfates or mixtures thereof;
from 1% to 12% by weight relative to the total weight of the formulation, in particular from 2% to 8% by weight of non-ionic surfactants, comprising:
a first non-ionic surfactant having a (C3-C7)alkyl chain;
a second non-ionic surfactant having a (C8-C20)alkyl chain;
the said formulation being a so-called concentrated formulation.

18. A formulation according to any one of claims 1 to 16, comprising:
from 0.2% to 2.5% by weight relative to the total weight of the formulation of at least one biocidal active substance, the active substance being a C1-C8 organic acid, in particular an alpha hydroxy carboxylic acid;
from 0.1% to 5% by weight relative to the total weight of the formulation, in particular from 0.3% to 2% by weight, of at least one anionic surfactant, in particular chosen from carboxylates, sulfates or mixtures thereof;
from 0.09% to 4% by weight relative to the total weight of the formulation, in particular from 0.1% to 2% by weight of non-ionic surfactants, comprising:
a first non-ionic surfactant having a (C3-C7)alkyl chain;
a second non-ionic surfactant having a (C8-C20)alkyl chain;
the said formulation being a ready to use formulation.

19. A formulation according to claim 18, wherein the first non-ionic surfactant represents from 0.009% to 1.8% by weight, in particular from 0.02% to 0.9% by weight relative to the total weight of the formulation.

20. A formulation according to any one of claim 18 or 19, wherein the second non-ionic surfactant represents from 0.08% to 2% by weight relative to the total weight of the formulation.

21. A formulation according to any one of claims 18 to 20, wherein the ready to use formulation is obtained by the diluting of a formulation as defined in claim 17 with water, in particular in a ratio: of 0.1% to 25% by weight (of the concentrated formulation in water), in particular of 0.5% to 10% by weight, more specifically of 0.5% to 5% by weight.

22. A formulation according to any one of the preceding claims, wherein more than 50% by weight, preferably more than 75% by weight, in particular more than 95% by weight of the ingredients of the formulation are derived from renewable sources.

23. A formulation according to any one of the preceding claims, characterised in that it is more than 95% biodegradable, in particular more than 98% biodegradable in accordance with the OECD 301 Guidelines.

24. The use of a biocidal formulation according to any one of claims 1 to 23, for cleaning and disinfecting inanimate hard surfaces, including surfaces that come into contact with food; for human hygiene, in particular for the sanitising and washing of hands; for cleaning and disinfecting animal teats before or after milking; and for washing and disinfecting fruits and vegetables.
